(19) United States
(12) Patent Application Publication
(10) Pub. No.: US 2017/0160353 A1
(43) Pub. Date: Jun. 8, 2017

(54) POSITIONING RADIO FREQUENCY RECEIVER COIL OF MAGNETIC RESONANCE IMAGING SYSTEM

(71) Applicant: SHENYANG NEUSOFT MEDICAL SYSTEMS CO., LTD., Shenyang (CN)

(72) Inventors: Huidong Gu, Shenyang (CN); Hongbing Hu, Shenyang (CN); Jianhua Shi, Shenyang (CN); Haobo Jin, Shenyang (CN)

(21) Appl. No.: 15/267,054

(22) Filed: Sep. 15, 2016

(30) Foreign Application Priority Data
Dec. 7, 2015 (CN) 201510891094.0

(51) Int. Cl. G01R 33/30; A61B 5/055; G01R 33/3415

(52) U.S. Cl. CPC G01R 33/307; G01R 33/3415; A61B 5/0555

(57) ABSTRACT

A method for positioning a radio frequency (RF) receiver coil in a Magnetic Resonance Imaging (MRI) system is provided. In one example method, a distance from a center of the RF receiver coil to one end of a support bed for carrying a subject and a distance from one end of the support bed to an imaging center of the MRI system may be obtained after locations of the RF receiver coil, the subject, and the support bed may be fixed. A distance from the center of the RF receiver coil to the imaging center of the MRI system may be obtained based on the obtained two distances. Moving the support bed a displacement equal to the distance from the center of the RF receiver coil to the imaging center of the MRI system may enable centering of the RF receiver coil on the imaging center of the MRI system.

### CROSS-REFERENCE TO RELATED APPLICATIONS

The present application claims priority to Chinese Patent Application No. 201510891094.0, filed on Dec. 7, 2015, the entire contents of which are hereby incorporated by reference for all purposes.

### BACKGROUND

This disclosure relates to positioning of a radio frequency (RF) receiver coil of a magnetic resonance imaging (MRI) system.

A MRI system may utilize RF energy to excite hydrogen atoms in a human body, so that the hydrogen atoms may resonate and generate magnetic resonance relaxation signals. The relaxation signals released from the hydrogen atoms may be collected by using a RF receiver coil, and the collected signals may be used to image the scanned area. Therefore, positioning the center of the RF receiver coil accurately on the imaging center of the MRI system, may improve the quality of the collected relaxation signals, thereby helping to obtain the best image quality.

A laser light may be configured to position the center of the RF receiver coil of the MRI system on the imaging center. Specifically, an operator may manually and slowly move a support bed which carries a subject, and during the moving, pay attention to the distance from the center of the RF receiver coil to the location of the laser light and implement manually positioning of the RF receiver coil.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, Magnetic Resonance Imaging (MRI), digital X-ray machine, ultrasound, Positron Emission Tomography (PET), Linear Accelerator (LINAC), and biochemistry analyser. Currently, NMS' products are exported to over 60 countries and regions around the globe, serving more than 5,000 renowned customers. NMS's latest successful developments, such as 128 Multi-Slice CT Scanner System, Superconducting MRI, LINAC, and PET products, have led China to become a global high-end medical equipment producer. As an integrated supplier with extensive experience in large medical equipment, NMS has been committed to the study of avoiding secondary potential harm caused by excessive X-ray irradiation to the subject during the CT scanning process.

### BRIEF DESCRIPTION OF DRAWINGS

The details of one or more embodiments of the subject matter described in this disclosure are set forth in the accompanying drawings and description below. Other features, aspects, and advantages of the subject matter will become apparent from the description, the drawings, and the claims. Features of this disclosure are illustrated by way of example and not limited in the following figures, in which like numerals indicate like elements.

FIG. 1 is a schematic diagram of a MRI system according to this disclosure;

FIG. 2 is a flowchart of a method for positioning a RF receiver coil of a MRI system according to this disclosure;

FIG. 3 is a schematic view of a support bed having a chute thereof provided with a linear resistor according to this disclosure;

FIG. 4 is a schematic view of a support bed and a RF receiver coil whose positions are fixed, according to this disclosure;

FIG. 5 is another schematic view of a support bed and a RF receiver coil whose positions are fixed, according to this disclosure;

FIG. 6 is a schematic diagram of a hardware configuration of an apparatus for positioning a RF receiver coil of a MRI system according to this disclosure;

FIG. 7 is a schematic block diagram of modules for control logic for positioning a RF receiver coil of a MRI system according to this disclosure.

### DETAILED DESCRIPTION

Examples of this disclosure may be applied to a MRI system, which may be mainly constructed by components such as a magnet 110, a gradient coil 120, a RF transmitter coil 130, a RF receiver coil, a support bed 140 carrying a subject, and so on. As shown in the right side of FIG. 1 is a schematic cross-section view of a MRI system. The magnet 110 may provide a local magnetic field intensity required for imaging. The gradient coil 120 may implement spatial-encoding of the subject to be imaged and may provide location information for the imaging operation. The RF transmitter coil 130 may be configured to excite the hydrogen atoms in the imaging area. The RF receiver coil may be attached to a to-be-scanned portion of the subject, and mainly configured to induce relaxation signals of the hydrogen atoms. The support bed 140 may be configured to support and position the subject, and be movable. In order to obtain a better imaging quality, the center of the RF receiver coil may be moved to the magnet center with the best magnetic field uniformity and best gradient linearity, that is, the imaging center of the MRI system, which will be described in the following examples, thereby achieving positioning of the RF receiver coil.

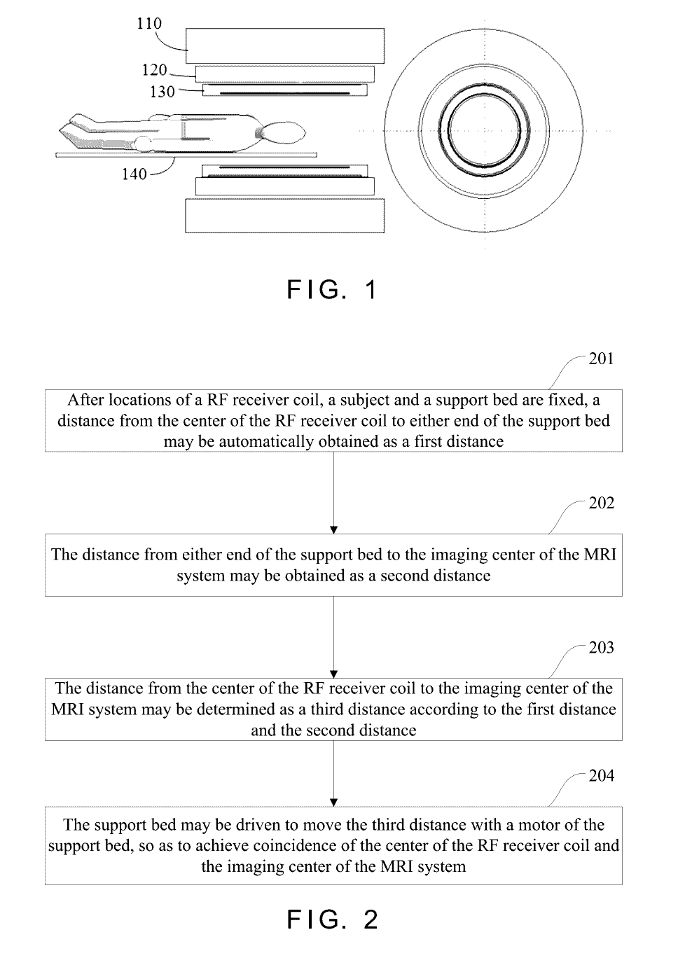

In an example, a method for positioning a RF receiver coil of a MRI system is provided. With reference to FIG. 2, FIG. 2 is a flowchart of a method for positioning a RF receiver coil of a MRI system according to this disclosure, and the method may comprise blocks 201-204.

In block 201, after locations of a RF receiver coil, a subject and a support bed are fixed, a distance from the center of the RF receiver coil to either end of the support bed may be automatically obtained as a first distance. Said either end of the support bed may be a heading end or a trailing end of the support bed.

In block 202, the distance from either end of the support bed to the imaging center of the MRI system may be obtained as a second distance.

In Block 203, the distance from the center of the RF receiver coil to the imaging center of the MRI system may be determined as a third distance according to the first distance and the second distance.

In block 204, the support bed may be driven to move the third distance with a motor of the support bed, so as to achieve coincidence of the center of the RF receiver coil and the imaging center of the MRI system.

When the subject and the RF receiver coil of the MRI system are placed properly, the RF receiver coil may be fixed by a Velcro strap. One end of the Velcro strap may be fixed to the RF receiver coil, and the other end may be fixed to the support bed carrying the subject. The one end of the Velcro strap that may be fixed to the support bed may slide freely in a chute of the support bed, and such fixation may satisfy the requirements for subjects having different heights or different clinical scanning portions.

In the examples of this disclosure, after the locations of the RF receiver coil, the subject, and the support bed may be fixed in the above-described manner, the distance from the center of the RF receiver coil to either end of the support bed (the heading end or the trailing end of the support bed) may be obtained, hereinafter referred to as a first distance.

In an example, in order to obtain the first distance, the MRI system may be modified, for example, at least the support bed of the MRI system may be modified. Specifically, a chute of the support bed may be provided with a linear resistor. For example, the linear resistor may be positioned on a side of the chute which may be near to the center of the support bed, or on a side of the chute which may be far from the center of the support bed, or even on both of the sides. Assume that the resistance per unit length of the linear resistor is Ru. Thus, in the case that the RF receiver coil connects via the strap with the chute of the support bed, the strap may be in junction with the linear resistor provided in the chute to form a resistor circuit. The resistance value at a location point where the strap may be in junction with the chute may be proportional to the displacement D from the location point to either end of the support bed. In other words, by measuring the resistance value Rm at the junction where the RF receiver coil may be in junction with the chute provided with the linear resistor, the displacement D (=Rm/Ru) from that junction to either end of the support bed may be determined.

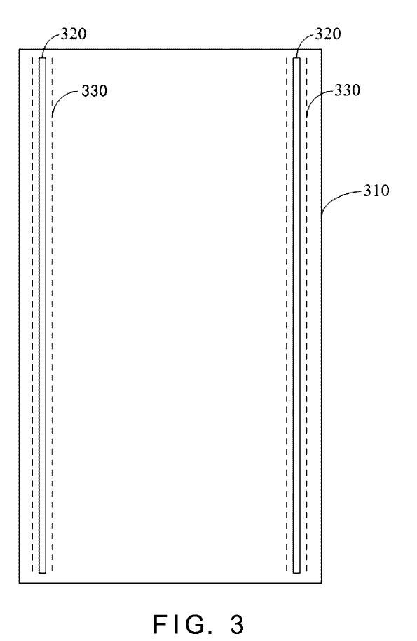

FIG. 3 is a schematic view of a support bed having a chute provided with a linear resistor, according to an example of this disclosure. The chutes 320 on both sides of the support bed 310 may be provided with a linear resistor 330. When any location point on the linear resistor 330 may be taken as a junction where the RF receiver coil may be in junction with the chute 320, the resistance value at that location point may be proportional to the distance from that location point to either end of the support bed 310. By calculating the ratio of the resistance value Rm measured at that location point with respect to the resistance value Ru per unit length of the linear resistor 330, the distance from that location point, i.e., the junction where the RF receiver coil may be in junction with the chute, to either end of the support bed 310 may be determined. Thereafter, based on the distances from the respective junctions where the RF receiver coil may be in junction with the chute 320 to either end of the support bed 310, the distance from the center of the RF receiver coil to either end of the support bed 310 may be calculated. This disclosure may not be limited in regard to the method for obtaining the resistance value of the linear resistor, for example, a multimeter may be used, or a constant electrical current source may be installed, so as to obtain the resistance value by measuring a voltage.

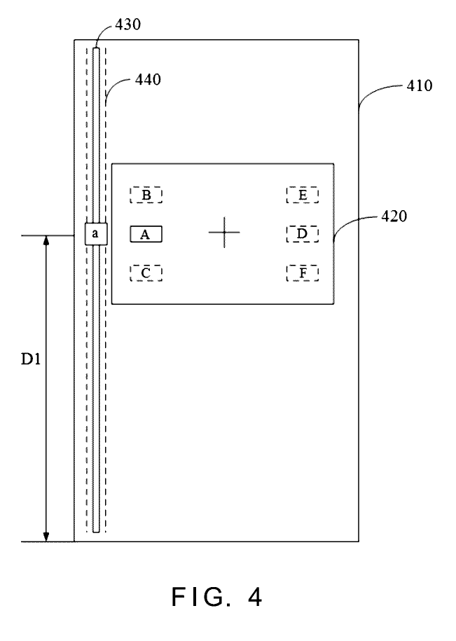

In an example of this disclosure, the RF receiver coil may be in junction via a strap with a location point in the chute provided with a linear resistor. FIG. 4 is a schematic view of a support bed and a RF receiver coil whose positions are fixed, according to an example of this disclosure. As shown in FIG. 4, the RF receiver coil 420 may be provided with two fixing points A, D, and the two fixing points A, D as well as the center of the RF receiver coil 420 may be equidistant from either end of the support bed 410 in the moving direction of the support bed. Further, the RF receiver coil may also comprise fixing points B, C, E, F for being fixed additionally. After the fixing point A of the RF receiver coil 420 is in junction via the strap with a location point “a” on the chute 430 provided with a linear resistor 440, the resistance value Ra at the location point “a” may be measured and the ratio of the resistance value Ra with respect to the resistance value Ru per unit length of the linear resistor 440 may be calculated. Then the distance D1 from the location point “a” to the trailing end of the support bed 410, i.e., the first distance, may be determined.

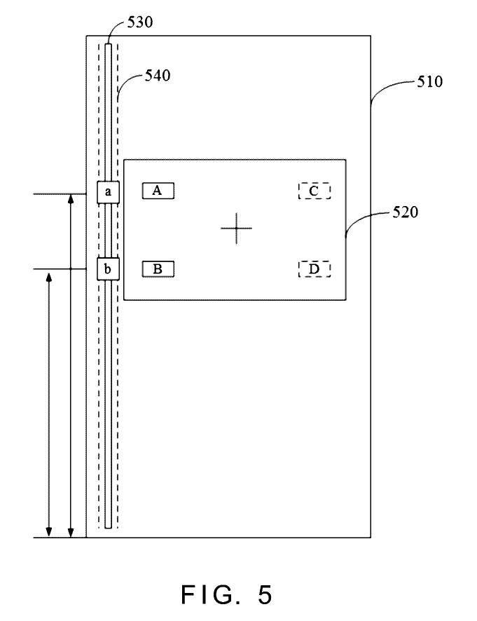

In another example of this disclosure, the RF receiver coil may be in junction via the strap with two location points in the chute provided with a linear resistor. FIG. 5 is a schematic view of a support bed and a RF receiver coil whose positions are fixed, according to an example of this disclosure. As shown in FIG. 5, the RF receiver coil 520 may be provided with at least four fixing points A, B, C, D, and the center of the RF receiver coil 520 may be the center of a rectangle defined by the four fixing points A, B, C, D. Further, the RF receiver coil 520 may also comprise other fixing points for being fixed additonally. After the fixing point A of the RF receiver coil 520 is in junction via the strap with a location point “a” on the chute 530 provided with a linear resistor 540, the resistance value Ra at the location point “a” may be measured and the ratio of the resistance value Ra with respect to the resistance value Ru per unit length of the linear resistor 540 may be calculated. Then the distance Da from the location point “a” to the trailing end of the support bed 510 may be determined. After the fixing point B of the RF receiver coil 520 is in junction via the strap with a location point “b” on the chute 530, the resistance value Rb at the location point “b” may be measured and the ratio of the resistance value Rb with respect to the resistance value Ru per unit length of the linear resistor 540 may be calculated. Then the distance Db from the location point “b” to the trailing end of the support bed 510 may be determined. Since the midpoint between the two location points “a”, “b” and the center of the RF receiver coil may be equidistant from the trailing end of the support bed 510 in the moving direction of the support bed, the average value of the distances respectively from the two location points “a”, “b” to the trailing end of the support bed 510 may be calculated and used as the first distance.

According to the above two examples of this disclosure, other approaches for obtaining the first distance may be further given and their description may be omitted here.

In addition, a MRI system may obtain the distance from either end of the support bed to the imaging center of the MRI system almost in real-time, and the distance may be referred to as a second distance hereinafter. Therefore, in examples of this disclosure, by using the first distance and the second distance, a distance, hereinafter referred to as a third distance, from the center of the RF receiver coil to the imaging center of the MRI system may be calculated.

In practice, if the first distance is the distance from the center of the RF receiver coil to the trailing end of the support bed, and the second distance is the distance from the trailing end of the support bed to the imaging center of the system, then the third distance may be obtained by subtracting the first distance from the second distance. Further, if the first distance is the distance from the center of the RF receiver coil to the heading end of the support bed, and the second distance is the distance from the heading end of the support bed to the imaging center of the system, then the third distance may be obtained by adding the first distance to the second distance. Further, if the first distance is the distance from the center of the RF receiver coil to the heading end of the support bed, and the second distance is the distance from the trailing end of the support bed to the imaging center of the system, then the third distance may be obtained by subtracting the length of the support bed from the sum of the first distance and the second distance. Further, if the first distance is the distance from the center of the RF receiver coil to the trailing end of the support bed, and the second distance is the distance from the heading end of the support bed to the imaging center of the system, then the third distance may specifically be obtained by subtracting the first distance from the sum of the second distance and the length of the support bed.

Finally, in the examples of this disclosure, after the distance from the center of the RF receiver coil to the imaging center of the MRI system may be determined, by driving from a motor of the support bed, the support bed may move a displacement equal to the third distance, so as to achieve automatic positioning of the RF receiver coil on the imaging center of the MRI system.

In the examples of this disclosure, the RF receiver coil may comprise a multi-channel body coil to scan the torso portion of the subject, may also comprise a brain coil for scanning the brain of the subject, and may further comprise a foot coil for scanning the feet of the subject.

According to the method for positioning a RF receiver coil of a MRI system in this disclosure, after locations of the RF receiver coil, a subject and a support bed may be fixed, the distance from the center of the RF receiver coil to either end of the support bed as well as the distance from either end of the support bed to the imaging center of the MRI system may be automatically obtained. Then the distance from the center of the RF receiver coil to the imaging center of the MRI system may be determined. Thus, by driving a motor of the support bed, the support bed may move a displacement equal to the distance from the center of the RF receiver coil to the imaging center of the MRI system, so as to achieve positioning the center of the RF receiver coil on the imaging center of the MRI system. After determining the distance from the center of the RF receiving coil to the imaging center of the MRI system, the motor of the support bed may be directly driven to automatically locate RF receiver coil. Compared with a manual positioning method by using a laser light, the method in the examples of this disclosure may improve positioning efficiency and save time of the positioning process.

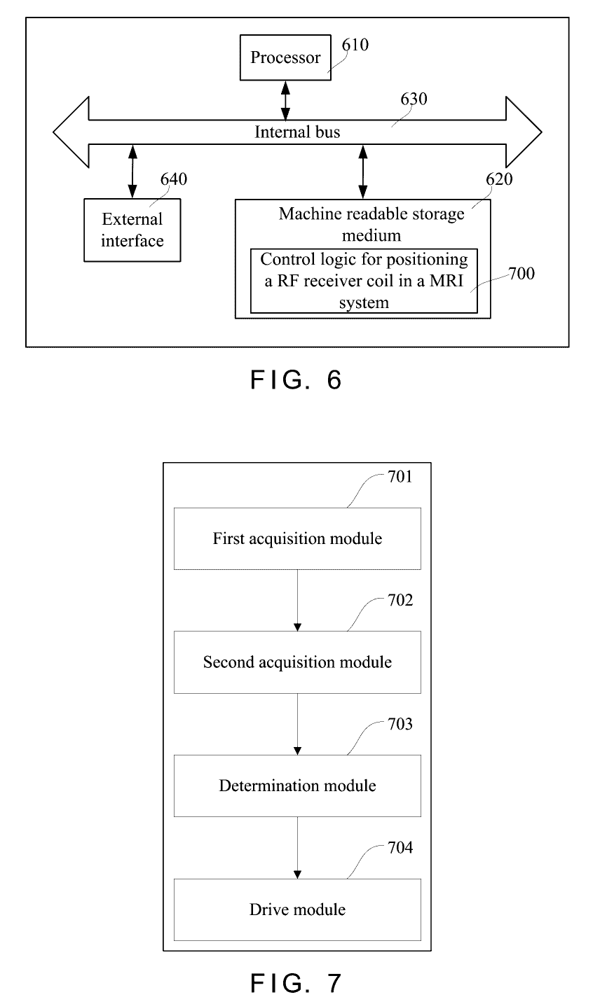

According to an example of this disclosure, an apparatus for positioning a RF receiver coil of a MRI system may also be provided. With reference to FIG. 6, the apparatus may comprise a processor 610 and a machine-readable storage medium 620. The processor 610 and the machine-readable storage medium 620 may be interconnected through an internal bus 630. In other possible example, the apparatus may further comprise an external interface 640, to enable communication with other components or devices such as the support bed.

In a different example, the machine-readable storage medium 620 may be RAM (Radom Access Memory), volatile memory, nonvolatile memory, flash memory, storage drives (such as hard drives), solid state drives, any type of storage disks (such as CD-ROM, dvd, etc.), or the like storage medium, or a combination thereof.

Further, the machine-readable storage medium 620 may store control logic 700 for positioning the RF receiver coil in the MRI system. As shown in FIG. 7, divided functionally, the control logic 700 may comprise the following blocks.

A first acquisition module 701 may be configured to automatically obtain a distance from a center of the RF receiver coil to one end of a support bed for carrying a subject as a first distance, after locations of the RF receiver coil, the subject and the support bed are fixed, wherein said one end of the support bed may be a heading end or a trailing end of the support bed.

A second acquisition module 702 may be configured to obtain a distance from one end of the support bed to an imaging center of the MRI system, as a second distance.

A determination module 703 may be configured to determine a distance from the center of the RF receiver coil to the imaging center of the MRI system as a third distance, according to the first distance and the second distance.

A drive module 704 may be configured to drive a motor of the support bed so as to move the support bed a displacement equal to the third distance, so as to achieve coincidence of the center of the RF receiver coil and the imaging center of the MRI system, that is, achieve positioning of the RF receiver coil.

In actual applications, the RF receiver coil may comprise a multi-channel body coil for scanning a torso portion of the subject.

According to an example, the support bed may be provided with two chutes respectively on two sides along the moving direction of the support bed, and at least one of the chutes may be provided with a linear resistor on its outer periphery, and a resistance value at any location point on the chute provided with the linear resistor may be measured. In this case, the first acquisition module 701 may calculate the distance from the center of the RF receiver coil to either end of the support bed by using the resistance value at the junction of the RF receiver coil and the chute.

Specifically, the RF receiver coil may be provided with at least two fixing points on both sides, and the two fixing points as well as the center of the RF receiver coil may be equidistant from either end of the support bed in the moving direction of the support bed. The fixing point on one side may be in junction via a strap with a location point on the chute provided with the linear resistor. In this case, the first acquisition module 701 may, based on the resistance value at the location point of the chute provided with the linear resistor, calculate the distance from the location point to either end of the support bed, as the distance from the center of the RF receiver coil to either end of the support bed.

In another example, the RF receiver coil may be provided with at least four fixing points, and the center of the RF receiver coil may be the center of a rectangle defined by the four fixing points, and the four fixing points may be respectively connected via a strap to total four location points on the chutes on both sides of the support bed. In this case, the first acquisition module 701 may, based on the resistance values at the two location points of the chute on one side, calculate the distances respectively from the two location points on the same side to either end of the support bed. Then, according to the distances respectively from the two location points on the same side to either end of the support bed, the distance from the midpoint between the two location points on the same side to either end of the support bed may be calculated, as the distance from the center of the RF receiver coil to either end of the support bed.

Taking a software implementation as an example, the following further describes how the apparatus for positioning a RF receiver coil in a MRI system may execute the control logic 700. In this example, the control logic for positioning a RF receiver coil may be understood as machine-executable instructions stored in a machine-readable storage medium 620. When the processor 610 on the apparatus for positioning a RF receiver coil in a MRI system executes the control logic 700, the processor 61 may perform the following operations by calling and executing the machine-executable instructions stored in the machine-readable storage medium 620.

The processor may obtain a distance from a center of the RF receiver coil to either end of a support bed for carrying a subject as a first distance, after locations of a RF receiver coil, a subject and a support bed may be fixed.

The processor may obtain a distance from either end of the support bed to an imaging center of the MRI system, as a second distance.

The processor may determine, according to the first distance and the second distance, a distance from the center of the RF receiver coil to the imaging center of the MRI system as a third distance.

The processor may make the support bed move the third distance, so as to achieve positioning the center of the RF receiver coil on the imaging center of the MRI system.

Said either end of the support bed may be a heading end or a trailing end of the support bed.

According to an example, the RF receiver coil may comprise a multi-channel body coil for scanning a torso portion of the subject.

According to an example, the support bed may be provided with two chutes respectively on two sides along the moving direction of the support bed, and at least one of the chutes may be provided with a linear resistor. In this case, the machine-readable instructions may cause the processor 610 to calculate the distance from the center of the RF receiver coil to either end of the support bed by using the resistance value at the junction of the RF receiver coil and the chute provided with the linear resistor.

Further, in a case where the RF receiver coil may be in junction via a strap with a location point of each of the chutes, wherein the location point and the center of the RF receiver coil may be equidistant from either end of the support bed in the moving direction of the support bed, when obtaining the distance from the center of the RF receiver coil to either end of the support bed, the machine-readable instructions may cause the processor to:

acquire a resistance value at the location point in the chute provided with the linear resistor, and

calculate the length from the location point to either end of the support bed as the distance from the center of the RF receiver coil to either end of the support bed based on the resistance value.

In another example, in a case where the RF receiver coil may be in junction via a strap with two location points in the chute on either side of the support bed, and the center of the RF receiver coil may be the center of a rectangle defined by the total four location points on the chutes on both sides of the support bed, when obtaining the distance from the center of the RF receiver coil to either end of the support bed, the machine-readable instructions may cause the processor to:

acquire resistance values at the two location points of the chute on one side of the support bed, wherein the chute may be provided with the linear resistor;

calculate the lengths respectively from the two location points of the chute to either end of the support bed based on the acquired resistance values; and

calculate the length from the midpoint between the two location points of the chute to either end of the support bed as the distance from the center of the RF receiver coil to either end of the support bed, according to the lengths respectively from the two location points of the chute to either end of the support bed.

The above are only examples of this disclosure and are not intended to limit the disclosure within the spirit and principles of this disclosure, any changes made, equivalent replacement, or improvement in the protection of this disclosure should contain within the range.

The methods, processes and units described herein may be implemented by hardware (including hardware logic circuitry), software or firmware or a combination thereof. The term ‘processor’ may be interpreted broadly to include a processing unit, ASIC, logic unit, or programmable gate array etc. The processes, methods and functional units may all be performed by the one or more processors; reference in this disclosure or the claims to a ‘processor’ should thus be interpreted to mean ‘one or more processors’.

Further, the processes, methods and functional units described in this disclosure may be implemented in the form of a computer software product. The computer software product may be stored in a storage medium and comprises a plurality of instructions for making a processor to implement the methods recited in the examples of this disclosure.

The figures are only illustrations of an example, wherein the units or procedure shown in the figures are not necessarily essential for implementing this disclosure. Those skilled in the art will understand that the units in the device in the example can be arranged in the device in the examples as described, or can be alternatively located in one or more devices different from that in the examples. The units in the examples described can be combined into one module or further divided into a plurality of sub-units.

Although the flowcharts described show a specific order of execution, the order of execution may differ from that which may be depicted. For example, the order of execution of two or more blocks may be changed relative to the order shown. Also, two or more blocks shown in succession may be executed concurrently or with partial concurrence. All such variations are within the scope of this disclosure.

For simplicity and illustrative purposes, this disclosure may be described by referring mainly to an example thereof. In the following description, numerous specific details are set forth in order to provide a thorough understanding of this disclosure. It will be readily apparent however, that this disclosure may be practiced without limitation to these specific details. In other instances, some methods and structures have not been described in detail so as not to unnecessarily obscure this disclosure. As used herein, the terms “a” and “an” are intended to denote at least one of a particular element, the term “includes” means includes but not limited to, the term “including” means including but not limited to, and the term “based on” means based at least in part on.

Throughout this disclosure, the word “comprise”, or variations such as “comprises” or “comprising”, will be understood to imply the inclusion of a stated element, integer or block, or group of elements, integers or blocks, but not the exclusion of any other element, integer or block, or group of elements, integers or blocks.

It will be appreciated by persons skilled in the art that numerous variations and/or modifications may be made to the above-described embodiments, without departing from the broad general scope of this disclosure. The present embodiments are, therefore, to be considered in all respects as illustrative and not restrictive.

1. A method for positioning a radio frequency (RF) receiver coil in a Magnetic Resonance Imaging (MRI) system, comprising:
obtaining a distance from a center of the RF receiver coil to one end of a support bed for carrying a subject as a first distance, after locations of the RF receiver coil, the subject and the support bed are fixed;
obtaining a distance from one end of the support bed to an imaging center of the MRI system, as a second distance;
determining, according to the first distance and the second distance, a distance from the center of the RF receiver coil to the imaging center of the MRI system as a third distance;
moving the support bed by the third distance to position the center of the RF receiver coil on the imaging center of the MRI system.

2. The method according to claim 1, wherein,
the RF receiver coil comprises a multi-channel body coil,
the multi-channel body coil is to scan a torso portion of the subject.

3. The method according to claim 1, wherein,
the support bed is provided with two chutes respectively on two sides along a moving direction of the support bed, and at least one of the chutes is provided with a linear resistor;
obtaining the distance from the center of the RF receiver coil to one end of the support bed, comprises:
acquiring a resistance value at a junction of the RF receiver coil and the at least one chute which is provided with the linear resistor; and
obtaining the distance from the center of the RF receiver coil to one end of the support bed is based on a ratio of the acquired resistance value with respect to a resistance value per unit length of the linear resistor.

4. The method according to claim 3, wherein,
the RF receiver coil is in junction with a location point of each of the chutes,
the location point and the center of the RF receiver coil are equidistant from one end of the support bed in the moving direction of the support bed;
obtaining the distance from the center of the RF receiver coil to one end of the support bed, comprises:
acquiring a resistance value at the location point of the at least one chute provided with the linear resistor,
determining a length from the location point to one end of the support bed as the distance from the center of the RF receiver coil to one end of the support bed by calculating a ratio of the resistance value with respect to the resistance value per unit length of the linear resistor.

5. The method according to claim 3, wherein,
the RF receiver coil is in junction with two location points of each of the chutes,
a midpoint between the two location points and the center of the RF receiver coil are equidistant from one end of the support bed in the moving direction of the support bed; obtaining the distance from the center of the RF receiver coil to one end of the support bed, comprises:
acquiring resistance values at the two location points of the at least one chute provided with the linear resistor,
determining distances respectively from the two location points to one end of the support bed by calculating ratios of the acquired resistance values with respect to the resistance value per unit length of the linear resistor,
determining the distance from the center of the RF receiver coil to one end of the support bed by calculating an average value of the distances respectively from the two location points to one end of the support bed.

6. The method according to claim 1, wherein one end of the support bed comprises a heading end or a trailing end of the support bed.

7. An apparatus for positioning a radio frequency (RF) receiver coil in a Magnetic Resonance Imaging (MRI) system, comprising a processor and a storage medium for storing machine readable instructions corresponding to control logic for positioning the RF receiver coil, where by executing the machine readable instructions, the processor is caused to:
obtain a distance from a center of the RF receiver coil to one end of a support bed for carrying a subject as a first distance, after locations of the RF receiver coil, the subject and the support bed are fixed;
obtain a distance from one end of the support bed to an imaging center of the MRI system, as a second distance;
determine, according to the first distance and the second distance, a distance from the center of the RF receiver coil to the imaging center of the MRI system as a third distance;
move the support bed by the third distance to position the center of the RF receiver coil on the imaging center of the MRI system.

8. The apparatus according to claim 7, wherein,
the RF receiver coil comprises a multi-channel body coil,
the multi-channel body coil is to scan a torso portion of the subject.

9. The apparatus according to claim 7, wherein, in case that the support bed is provided with two chutes respectively on two sides along the moving direction of the support bed, and at least one of the chutes is provided with a linear resistor, when obtaining the distance from the center of the RF receiver coil to one end of the support bed, the machine-readable instructions further cause the processor to:
acquire a resistance value at a junction of the RF receiver coil and the at least one chute which is provided with the linear resistor,
obtain the distance from the center of the RF receiver coil to one end of the support bed, based on a ratio of the acquired resistance value with respect to a resistance value per unit length of the linear resistor.

10. The apparatus according to claim 9, wherein, in case that the RF receiver coil is in junction with a location point of each of the chutes, the location point and the center of the RF receiver coil are equidistant from one end of the support bed in a moving direction of the support bed, when obtaining the distance from the center of the RF receiver coil to one end of the support bed, the machine-readable instructions further cause the processor to:
acquire a resistance value at the location point of the at least one chute provided with the linear resistor,
determine a length from the location point to one end of the support bed as the distance from the center of the RF receiver coil to one end of the support bed by calculating a ratio of the resistance value with respect to the resistance value per unit length of the linear resistor.

11. The apparatus according to claim 9, wherein, in case that the RF receiver coil is in junction with two location points of each of the chutes, and a midpoint between the two location points and the center of the RF receiver coil are equidistant from one end of the support bed in the moving direction of the support bed, when obtaining the distance from the center of the RF receiver coil to one end of the support bed, the machine-readable instructions further cause the processor to:
acquire resistance values at the two location points of the at least one chute provided with the linear resistor;
determine distances respectively from the two location points to one end of the support bed by calculating ratios of the acquired resistance values with respect to the resistance value per unit length of the linear resistor;
determine the distance from the center of the RF receiver coil to one end of the support bed by calculating an average value of the distances respectively from the two location points to one end of the support bed.

12. The apparatus according to claim 7, wherein one end of the support bed comprises a heading end or a trailing end of the support bed.
